(19) United States
(12) Patent Application Publication
(10) Pub. No.: US 2007/0241018 A1
(43) Pub. Date: Oct. 18, 2007

(54) Personally-me wallet/pouch

(76) Inventor: Shirley Forte, Country Club Hills, IL (US)

Correspondence Address: SHIRLEY FORTE, 5001 WEST 180TH STREET, COUNTRY CLUB HILLS, IL, 60478, US

(21) Appl. No.: 11/731,089

(22) Filed: Mar. 31, 2007

Related U.S. Application Data
(60) Provisional application No. 60/787,824, filed on Mar. 31, 2006.

(51) Int. Cl. A45C 3/06; A61B 19/02; A45C 1/02

(52) U.S. Cl. 206/438; 150/112

(57) ABSTRACT

The Personally-Me menstrual wallet/pouch comprising: a five pocket unit which has two pieces of fabric strips, sewn together to form a small wallet/pouch. Inside the are individually sewn pockets used to carry the following female sanitary items: panty liner, wet wipe, tampon, sanitary napkin and calendar. The wallet/pouch if the approximate size of a small hand size wallet. When fully opened horizontally, the wallet is approximately 12 to 13 inches wide, with a height of approximate 5 to 6 inches. The top flap of the wallet/pouch's has a fabric strip that folds over the bottom half to form the wallet. Each side of the wallet has a zipper, so when fully opened all pockets can easily be seen at one time. When the top of the wallet folds over the bottom half, the wallet/pouch is additionally secured in place by two Velcro strips connecting one (located on inside top half of the flap) to the other (located on the outside bottom half of the wallet/pouch). With both sides of the wallet/pouch zipped, it then forms a deep pouch like purse creating even more storage space. The Personally-Me wallet/pouch comprises: an individual pocket made with a lace vinyl and a clear see through plastic for convenient viewing of all menstrual supplies. The wallet/pouch also comprises a calendar to calculate menstrual dates. The pockets are sewn in a horizontal row and attached to fabric backing of the wallet/pouch. All pockets have a pair of side edges, a bottom edge and a top edge. The top edge has the opening to place the feminine hygiene items into the pocket.

The present invention is a hand held fabric wallet/pouch consisting of five pockets. These five pockets are constructed and sewn to hold the following five menstrual supply items in a small fabric wallet/pouch: 1. panty liner, 2. wet wipes, 3. tampons, 4. sanitary napkins, and 5-a calendar. The wallet itself is made of fabric than can easily be hand or machine-washed. The wallet/pouch front outer covering is designed to show a variety embroidered or computerized names and other artwork used on fabrics.

Both sides of the pouch have an attached zipper. The zippers are used to fully open and close the wallet. When the wallet/pouch if fully opened, a full view of all items is available and accessible for use. When fully closed the contents of the wallet/pouch are secure and the entire storage area then becomes an enclosed pouch with additional storage space available.

To form the style of a wallet, there is a flap that folds over the bottom section of the wallet and it is held together with two strips of Velcro. The Velcro is used to give the wallet/pouch even more security when wallet is completely full. The wallet/pouch is designed to carry one or two panty liners, one or two wipes, one tampon, one or two sanitary napkins and one calendar. All pockets except the tampon pocket are have rectangle shapes. The tampon's pocket has a long cylinder shape that enables a tampon to easily slide in for storage. The panty liner, tampon, and sanitary pockets are all made of lace vinyl. By using the vinyl, the wallet/pouch can easily be wiped clean with a wet towel until is later washed.

### BACKGROUND OF THE INVENTION

Woman of various, religions, color and races have struggled with the dilemma of Finding an appropriate spot to keep menstrual items such as tampons and sanitary napkins and something to clean with if nothing available in a public washroom. Also, there's the question of keeping up with due dates every monthly cycle. This has proven especially difficult when it comes to pre-teen girls.

1. Field of Invention

The present invention relates to the storage of menstrual products and supplies for woman and especially pre-teen and teenage girls. It relates to the storage of menstrual supplies of panty liners, wet wipes, tampons, sanitary napkins and a paper calendar. It relates to a carry case for these menstrual supplies for pre-teen, teen-age, and older woman. female wallet formed with five pockets, which are especially useful in holding, storing and discretely carrying feminine menstrual products. It relates to the use and availability of a calendar, to aid to with the calculating of a woman's future monthly menstrual dates. It relates to an all in-one wallet, which can always be readily available for use by woman of all ages.

2. Description of Related Art

There are patents that deal with this is these issues but all are on a limited basis. For example, U. S. Pat. No. 6,076,195 to Klein discloses and undergarment having a series of pockets provided on a front side thereof. U.S. Pat. No. 6,772,446 to Black shows an undergarment with one or more pockets. Pockets that wrap around the sides of the undergarment to surreptitiously store a sanitary pad. The undergarment with side pockets provided an article of apparel developed for the purpose of permitting women to have a sanitary pad placed adjacent to her body without being seen. Also, U. S. Pat. No. 5,988,386 to Morrow references a rigid compartment unit for menstrual supplies which has additional strings and attachment for sanitary napkins, tampon plungers and a disposal unit.

In the past, the prior art has traditionally provided purses, handbags and various other carrying cases for sanitary pads usually being located in a small pocket located as an attachment of the entire purse, causing difficulty to access throughout the movement of everything else in the purse. These traditional purses or handbags only allow a small limited space for one or two items to be used. If there are numerous pockets of a purse or handbag, they are all in scattered location.

In this regard, the Personally-Me wallet/pouch will eliminate the many spaces needed in a traditional purse or bag and will give flexibility of carrying and storage regardless of the purse, purse size or other clothing worn by woman. There are no hidden undergarment to use or conceal. The wallet/pouch is a simple light-weighted carrying unit which is easy to use, clean, wash and re-use again every month.

### SUMMARY OF INVENTION

Many times menstrual flows occurs unexpectedly, but in any case there is a need for the menstrual supplies to be available during a planned or unplanned occasion.

It is the object of the Personally-Me wallet/pouch to assist in solving the problems Associated with carrying menstrual supplies during those planned and unplanned occasions.

It is the object of the Personally-Me wallet/pouch to hold the panty liner, wet wipe, tampon, sanitary napkin and a calendar in one area.

It is the object of the invention to provide a storage unit that is small enough to be carried in one hand, but still contain all supplies needed during a menstrual cycle. The wallet can be placed inside a traditional large or small purse, handbag, backpack, gym bag, pants or skirt pocket, or merely held as a stand along wallet. It is the object of the invention to resemble a traditional wallet and yet function as a special storage unit for personal menstrual supplies without the knowledge of anyone except the woman carrying it.

It is the object of the invention to include a combination of colors that helps it blend well with any fashion of clothing, if carried and not place in a purse. With the availability of the pouch, menstrual items can be stored ahead of time with every needed in one place, and ready to go. This is extremely useful to mothers of pre-teen girls and many women. The wallet can be carried within a purse or backpack daily and used whenever the need arises.

It is an the object of the invention to provide a carrying case for menstrual supplies that can always be available for transfer from one purse to another or from purse to gym or backpack in one step.

It is the object of the invention to protect the menstrual supplies from damage and contamination of other items in a purse.

It is the object of the invention to provide a much easier and convenient and advance way method for storing and carrying menstrual supplies.

It is the object of the invention to make it easier for pre-teen girls not to forget needed supplies when needed at school or various sport events.

It is the object of the invention to make it easier for the mothers of pre-teen girls to explain and note the days of each month when supplies will most likely be used. It is the object of the invention to eliminate the embarrassment of young girls who have unexpected periods during class or other events away from home.

It is the object of the invention to create a desire to always want to keep the pouch available by placing their name or theme of interest on the wallet/pouches. This invention relates to a wallet style purse that also takes on the feature of a pouch purse when closed. The wallet is small enough to fit into any size purse, backpack, or gym bag for planned or immediate emergency use. It houses all needed menstrual items in one fabric wallet style pouch which is washable and reusable on a monthly basis. A variety of products, bags and other garments are in use to carry feminine products to be used with the onset of menstrual periods. Since the typical packaging of sanitary items can be soiled, damaged or completely broken with mixed with other items found in woman purses, the wallet provide its own private and secure area away from all other items placed within a woman's purse, pre-teen and teenage backpacks and even gym bags. Many women use one types of feminine products at one time or another. Tampons are packaged in cylinder or tube like appearance.

The problem of emergency menstrual incidents has proven to be a problem for woman of all ages. It has especially been troublesome for young pre-teen girls. More and more young girls are starting their menstrual cycles as young as age 9. Many of these young girls show difficulty and embarrassment when experiencing these menstrual emergencies.

### BRIEF DESCRIPTION OF THE DRAWINGS

FIG. 1 is a perspective view of a wallet size storage unit according to the present invention and showing wallet/pouch fully closed with a front view of wallet.

FIG. 2 is a full view of the wallet/pouch when totally opened. This view shows the individual pocket as seen when the wallet/pouch is open and ready for storage of menstrual supplies. Pockets shown are numbered 1 through 5 as the following:
Pocket #1—is the pocket that stores a panty liner.
Pocket #2—is the pocket a see through vinyl square pocket that stores a wet wipe
Pocket #3—is a long cylinder shaped pocket that stores a tampon
Pocket #4—is the pocket that stores a sanitary napkin.
Pocket #5—is a second clear view pocket that stores a custom size 12-month Calendar.

FIG. 3 shows how pocket #2—(the wet wipe pocket) is overlapping pocket #1(the panty liner pocket).

FIG. 4 shows the view of pocket # 3,4,and 5—the calendar pocket.

FIG. 5 shows the angle and position of the wallet/pouch when zipped. This views shows how when zipped and closed the calendar automatically angles itself in an upright position for easy viewing and notations. FIG. 5 also shows the position of the two attached Velcro strips which adds additional security to the opening and closing of the wallet/pouch.

FIG. 6 is a closer view of the panty liner and wet wipe pockets (pockets 1 and 2)

FIG. 7 is a closer view of the Tampon pocket (pocket #3)

FIG. 8 is a closer view of the sanitary napkin pocket (pocket #4)

FIG. 9 is a closer view of the calendar pocket without the insertion of the calendar.

FIG. 10 is a view of the wallet/pouch when closed by zipping from bottom to top. With both sides zipped a deep pouch with additional space is formed in the wallet. This additional pouch space continues to be available even when all menstrual supplies are stored in pockets. One of each item will not make the pouch seem too bulky.

FIG. 10 also shows the position of the interior Velcro strip (just above the edge of the calendar pocket) will attach itself to the second Velcro strip directly below the opening of the wallet/pouch.

FIG. 11 shows the position of the two zippers at the bottom of the pouch when it is ready to be opened for use.

FIG. 12 shows the back view of the fully opened pouch before it is zipped into a pouch and folded into a wallet.

FIG. 13 is a closer view of FIG. 12. It shows the stitching of the Velcro, the vertical position of the rectangle shaped wallet/pouch, the middle positioning of the two sewn zippers. It also show about 75% of the pouch is closed with the two zipper and the other 25% (which is the flap from the calendar pocket) folds over the 75% of pouch to form a wallet style carrying case for menstrual supplies.

### DETAILED DESCRIPTION OF PREFERRED EMBODIMENTS

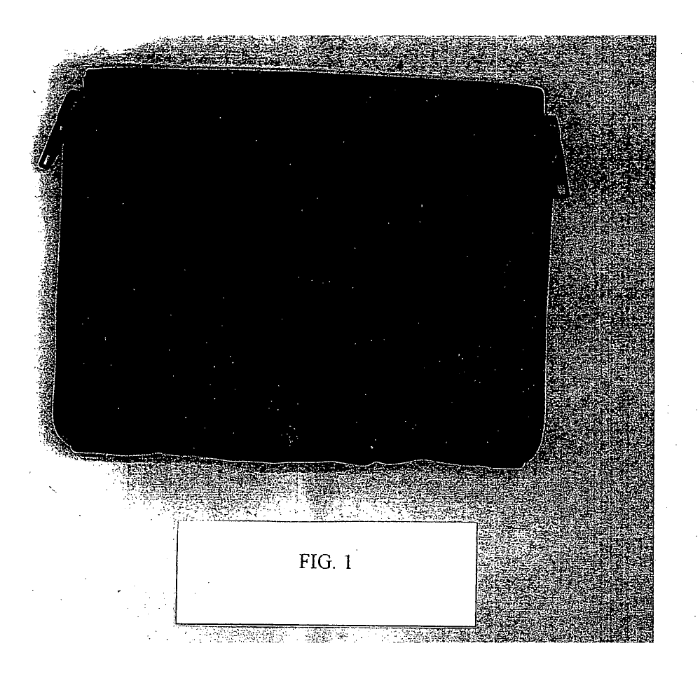

The present invention is a wallet used to store and carry menstrual supplies for woman of all ages. The wallet/pouch is made up of two equaled sized strips of fabric with an additional thin layer of fabric sewn between the two. The thin middle sheet of fabric is used to maintain the combined stability of the two outer pieces when sewing them together. The outside raw edges of the wallet and the zippers are all sew together on the wrong side and then turned inside out creating a clean-finished unit, as shown in FIG. 1 With pocket #2, the wet wipe and smallest of all five pockets if is placed over the bottom half of pocket 1. The #2 pocket covers half the length of pocket one. Pocket 2 is sewn directly into the side seams of pocket 1. The wet wipe pocket is designed to receive a single wet wipe that is contained in its own commercial packaging.

FIG. 2 show the order of positioning of all supplies when placed into their respective pocket. After pocket I and 2, you can see the cylinder pocket for the tampon. The arrow in FIG. 2 shows the area of opening for each of the remaining pockets. Pockets 1,2,3,and 4 are made of a rubberized/vinyl type lace design. Pockets 1,2,3, and 4 were given a flowering pattern to slightly obscure the view of contents if opened in a public area, but it's still open enough for women to always know what item she is looking at in the wallet/pouch.

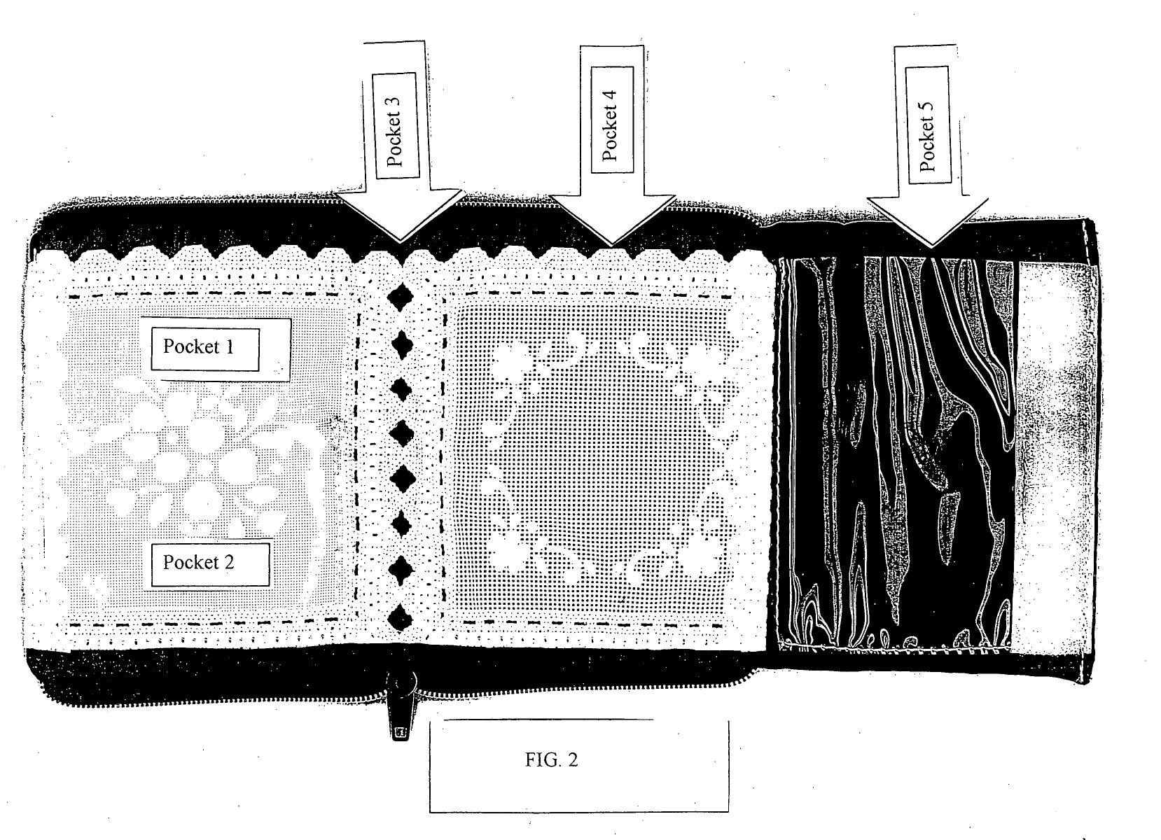

The #1 and #2 pockets shown in FIG. 3 show a clearer version of how and where the separation of the two pockets begins and ends. The content of one item never comes in contact with the other. In this view the left and right sides of the wallet have been folded back to show a clearer picture of the two pockets relationship to each other. The panty liner comes in a small thin commercial package can be placed into its pocket with ease. The positioning of all pockets within the wallet/pouch is created to give an ease of removal of items by most woman when changing in a toilet area. FIG. 2 also shows the outstretched width of the unit. Because the wallet/pouch opens completely, it enables a woman to clearly see and easily choose the immediate menstrual item needed. FIG. 2 shows the stitching of the pockets that flows from one to the other and yet, still remains separate items.

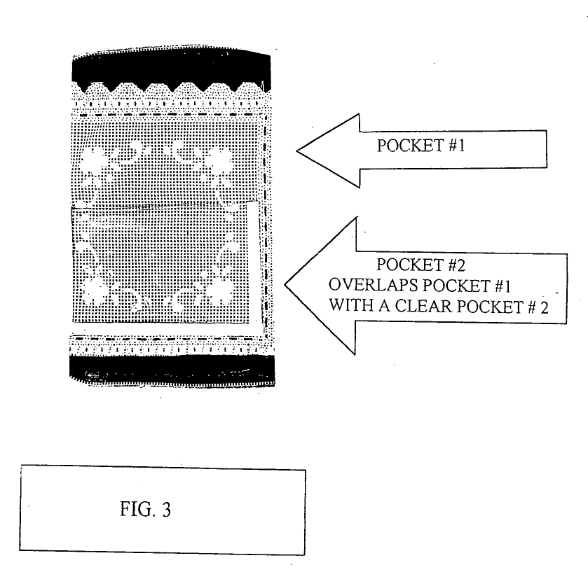

FIG. 4 shows another view of the tampon, sanitary napkin and calendar pocket. It also shows the view of the calendar when wallet is opened and resting in its horizontal position. The calendar displays a view of one month at a time. The clear plastic pocket protects the calendar from dirt and grime. The calendar itself is a group of lightweight single sheets stacked together and placed together inside the calendar through its top pocket entrance.

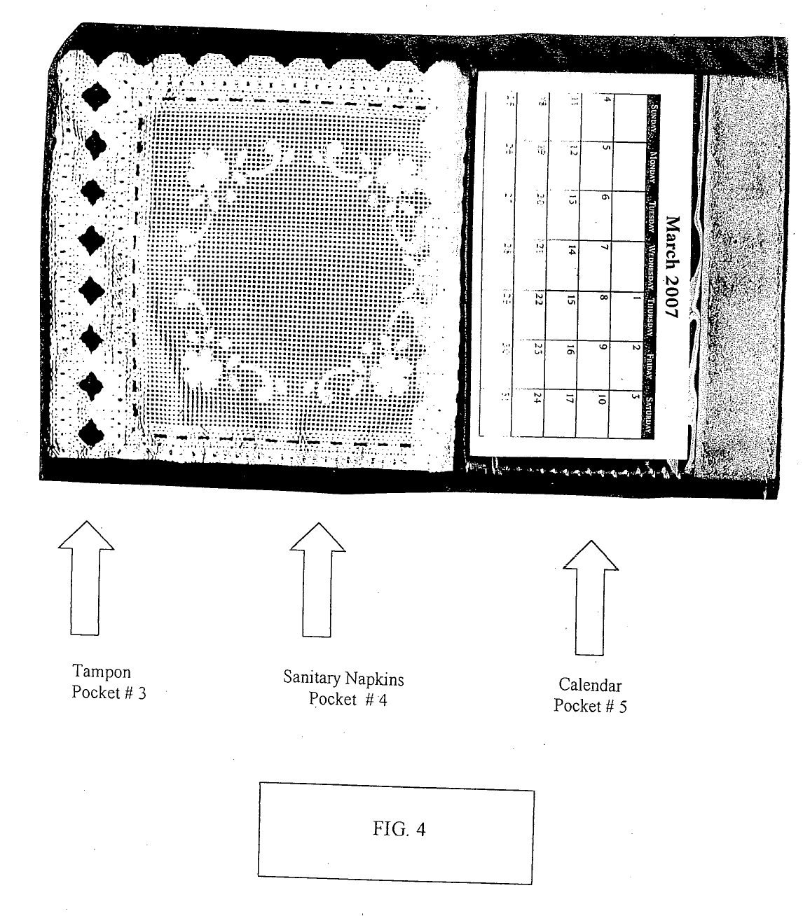

FIG. 5 shows the front view of the wallet/pouch with the top flap raised and showing the calendar as it appears when opened in its normal upright position.

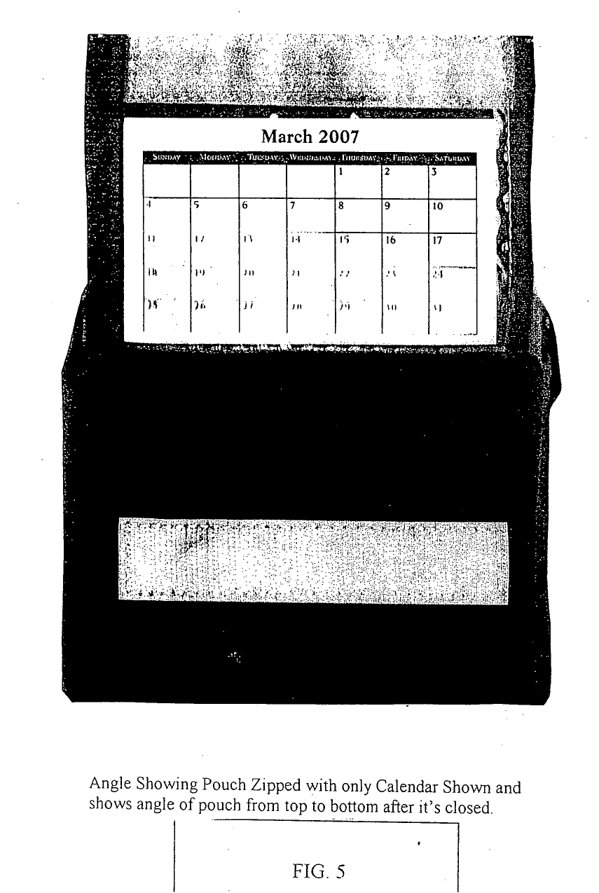

FIG. 6 shows a closer view of pocket 1 and 2 and shows how the two zippers separate on each end when the pouch if opened.

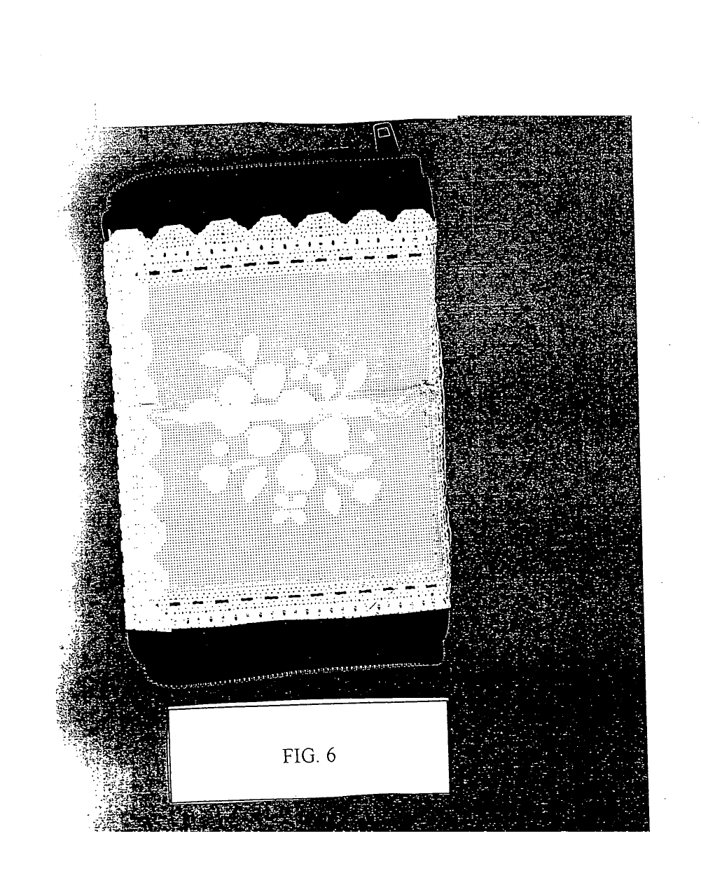

FIG. 7 shows both sides of the wallet/pouch folded back to get a closer view of cylinder pocket for a tampon. This pocket also has periodically diamond shaped hole along the vertical pocket. The diamond openings in the tampon pocket helps with the movement and ease of insertion of the tampon into its pocket.

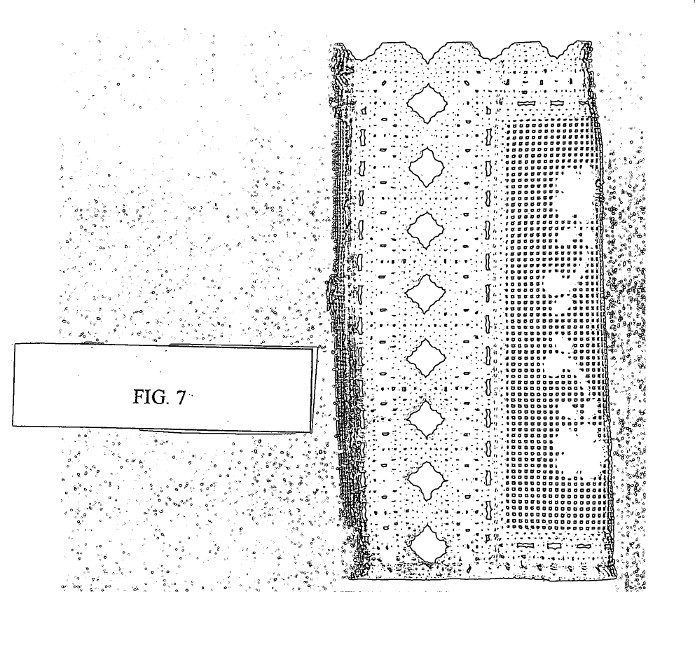

FIG. 8 is another closer view of the sanitary napkin pocket. This pocket is designed to hold one or two sanitary napkins. This is also another view of the zipper and pocket when shown from another angle.

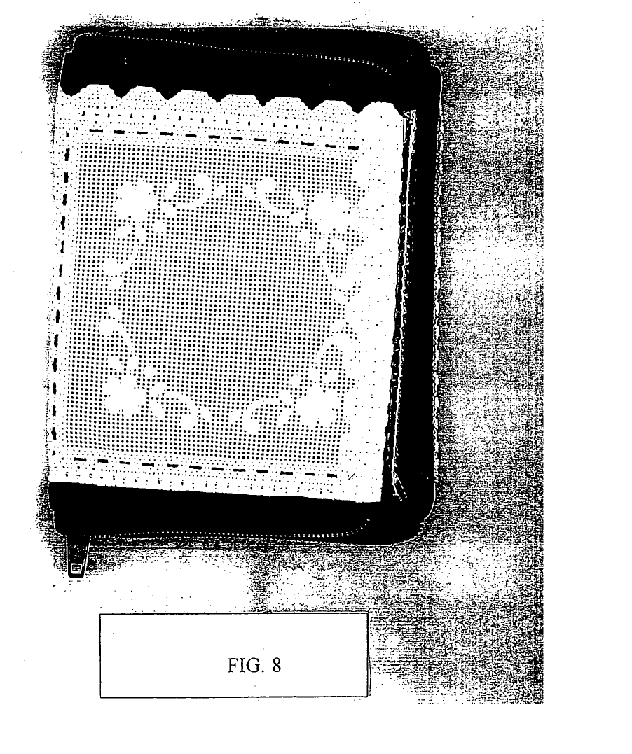

FIG. 9 shows a close up view of the calendar pocket. Both sides and bottom edge of pocket is sewn (as with other pockets) leaving a top opening for the calendar. Calendar size has been computer customized to fit freely inside the calendar pocket. The pocket itself is made up of a clear plastic, thus enabling the calendar to be viewed easily when opened. A Velcro strip is attached to one side of the pocket. This strip attaches to another to create a sturdy and tighter fastener for the wallet/pouch. The calendar shows each month on a single sheet of preprinted paper. The day of each month has ample amount of space, for women or pre-teen girls to note their next monthly menstrual date. The dates can be calculated from January through December. The loose-leaf calendar pages are the only item that automatically comes with the wallet/pouch. The customized calendar sized for the wallet/pouch can be re-ordered as needed. The pouch is designed to accept most traditional menstrual supplies.

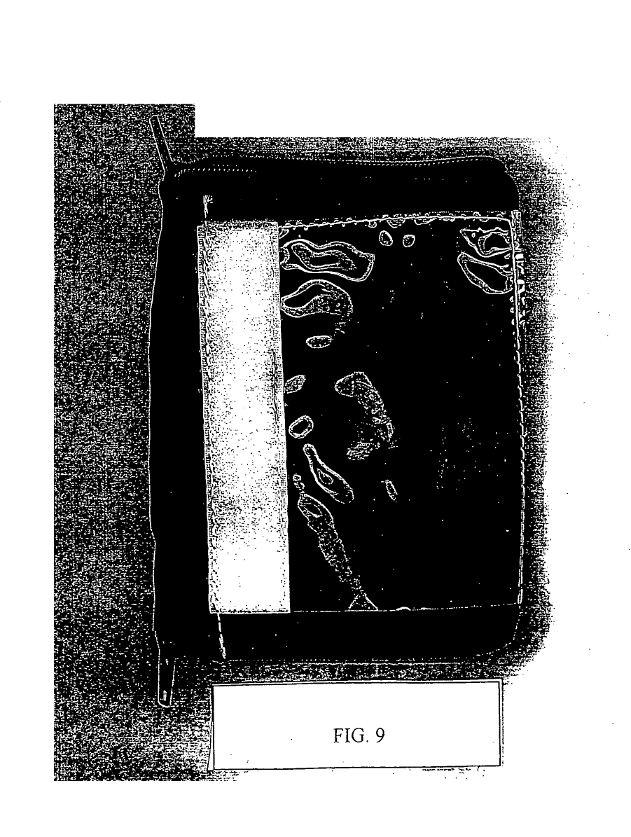

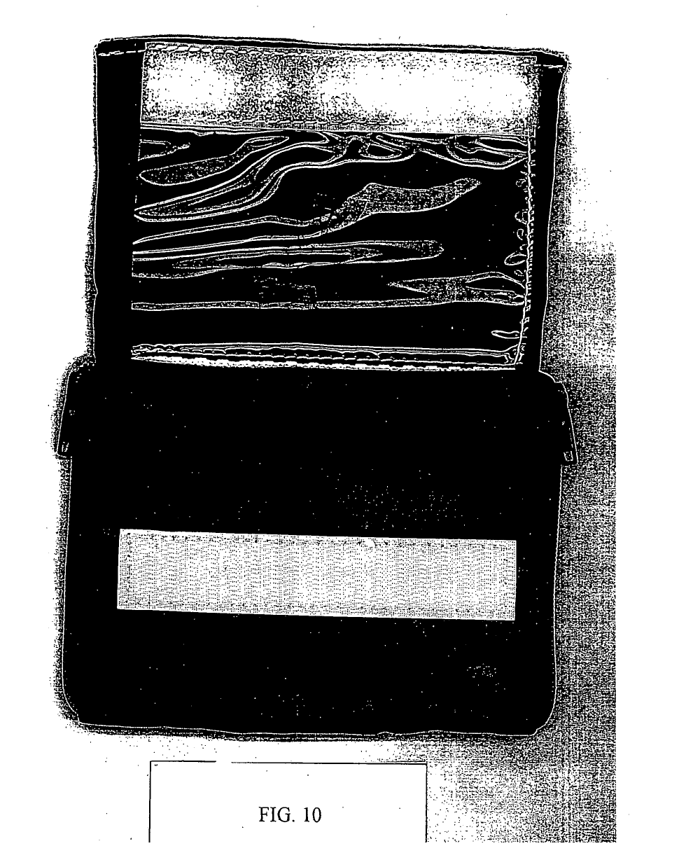

FIG. 10 is an opened front view of the wallet/pouch with its overlapping flap placed in an upright position. Also, in FIG. 10 the two side zippers are shown in a up position.

When the zippers of the wallet/pouch are in this upright position; it forms the deep pouch which creates even more space for items, if needed. The zippers are inter-locking zippers (this means that the zipper lock in position when they are zipped up) This use of this commercial style zipper insures that the sides of the pouch will not slide down or split when pouch is used or if it's ever stuffed full. The wallet/pouch can be carried with few supplies or until it is bulky, whatever the preference, the wallet pouch will not split or break apart. In the section of FIG. 10 there is also a closer view of the top and bottom Velcro strips that adds the additional strength for closure of the wallet/pouch.

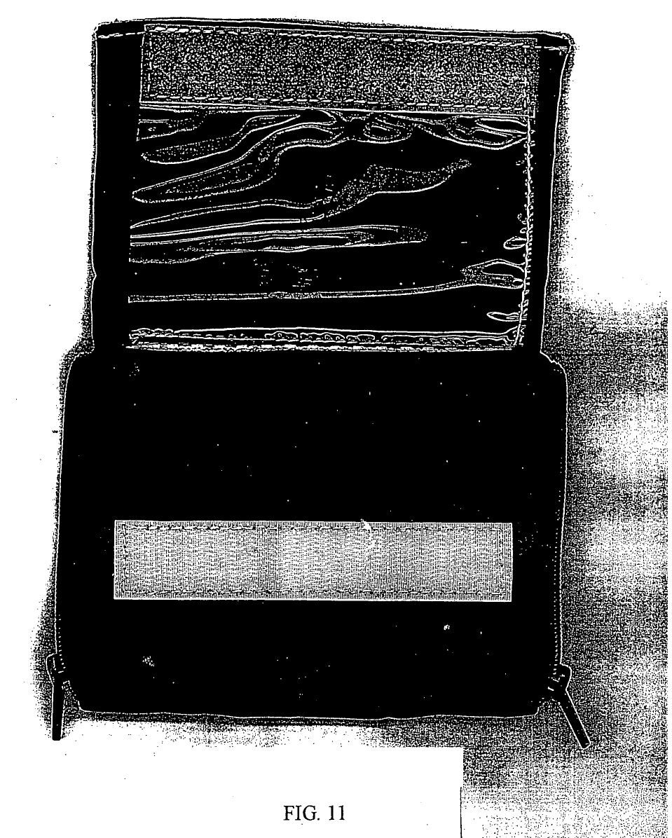

FIG. 11 shows the same pouch found in FIG. 10 except the zippers are now show in the down position. When both zippers are pulled down to the bottom position of the wallet/pouch, it now in a position to be opened with contents still in tack.

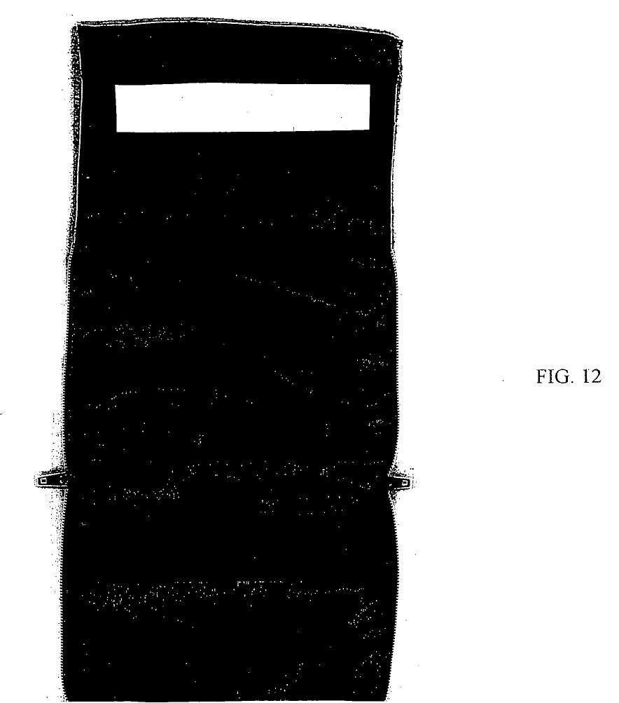

FIG. 12 shows the back angle of an opened wallet/pouch shown at a horizontal view.

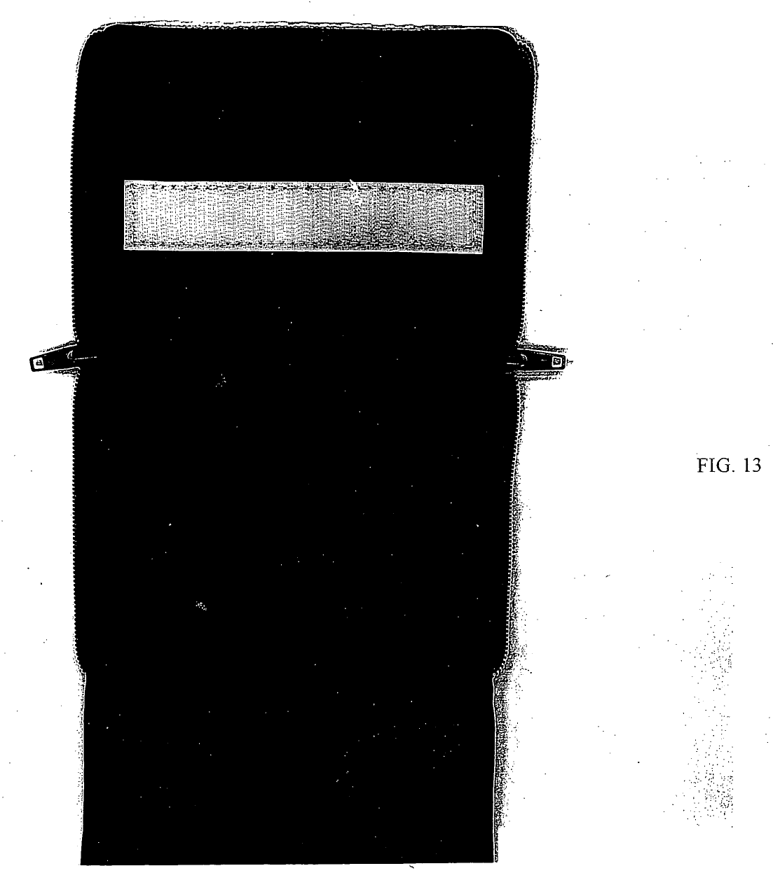

FIG. 13 shows the same back view of an opened pouch as FIG. 12, except the wallet/pouch is now shown as vertical view.

I claim:

1. The Personally-Me storage wallet/pouch comprising of five individual And separately sewn pockets for storage of store purchased panty liner, wet wipe, tampon, sanitary napkin and calendar, all sewn in a continuous horizontal row and attached to a fabric backing (backing which can also be made with a silk, cotton, leather or any other fabric) with the wet wipe and calendar pockets made of a clear plastic, for easy viewing, and the panty liner, tampon, and sanitary napkin pockets made of a lace vinyl.

2. The Personally-Me menstrual wallet/pouch comprising: two identical sized, The exterior and interior piece of fabric strips, sewn together to create a foldable hand carried fabric wallet that creates another deep storage area when closed, it's approximately 10 to 11 inches wide when unfolded in its unzipped and fully opened horizontal position and approximately 4 inches by 5 inches when completely closed and zipped back up in a vertical position for easy insertion into a larger purse, book or gym bag.

3. The top flap of the wallet/pouch fabric strip folds over the bottom half to form the wallet appearance. Each side of the wallet has a zipper, so when zipper is fully pulled down and opened all pockets and contents can easily be seen at one glance. Also, When the top flap of the wallet folds over the bottom half; the wallet/pouch is additionally secured in place with two Velcro strips connecting one (located on inside top half of the flap) to the other (located on the outside bottom half of the wallet/pouch). When both sides of the wallet/pouch are zipped up and closed; it then forms a deep pouch creating even more space and security of contents due to any type of rigorous handling.

4. The wallet/pouch according to claim 1, the use of the clear plastic and lace for pockets helps to limit the buildup of stains on the interior walls of the wallet/pouch, makes it easy to wipe clean with a wet towel and yet durable enough to be machine washed and air dried for a continuous clean and fresh smell for use every month.
